(19) United States
(12) Patent Application Publication
(10) Pub. No.: US 2024/0308966 A1
(43) Pub. Date: Sep. 19, 2024

(54) CHIRAL AMINE-SQUARAMIDE COMPOUND BASED ON SPIROBIINDANE SKELETON, PREPARATION METHOD AND USE THEREOF

(71) Applicant: ZHEJIANG UNIVERSITY, Hangzhou (CN)

(72) Inventors: Xufeng LIN, Hangzhou (CN); Zhao HAN, Hangzhou (CN)

(21) Appl. No.: 18/676,514

(22) Filed: May 29, 2024

Related U.S. Application Data
(63) Continuation of application No. PCT/CN2023/114140, filed on Aug. 22, 2023.

(30) Foreign Application Priority Data
Sep. 15, 2022 (CN) 202211124671.X

(51) Int. Cl. C07D 225/04; C07D 403/06

(52) U.S. Cl. CPC C07D 225/04; C07D 403/06

(57) ABSTRACT

A chiral amine-squaramide compound based on a spirobiindane skeleton, a preparation method and use thereof provided. The chiral amine-squaramide is an enantiomeric compound having the general formula (I) and is useful for asymmetric catalytic organic reactions with good catalytic activity and enantioselectivity. The chiral amine-squaramide compound is obtained by preparation of compound of formula (I) through addition of a compound of formula (II) with squaric acid ester. The synthetic reaction route is simple, and it is easy for large-scale application. The novel chiral amine-squaramide organocatalyst has economic utility and promising industrial applications.

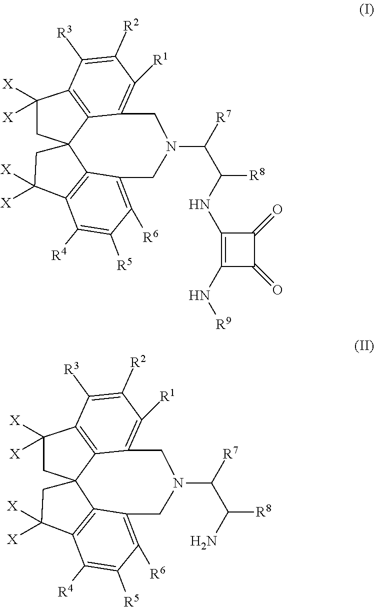

### CROSS-REFERENCE TO RELATED APPLICATIONS

This application is a National Stage of International Application No. PCT/CN2023/114140, filed Aug. 22, 2023, which claims priority to Chinese Patent Application No. 202211124671X, filed on Sep. 15, 2022, both of which are hereby incorporated by reference in their entireties.

### TECHNICAL FIELD

The present application relates to the field of asymmetric synthesis catalysts, and more particularly to a chiral amine-squaramide compound based on a spirobiindane skeleton, a preparation method and a use thereof.

### BACKGROUND

One of the core scientific problems of asymmetric catalysis is the development of new highly efficient chiral catalysts. Although the design synthesis of chiral catalysts has been rapidly developed, there are still problems including limited applicability of the catalyst, high dependence on the reaction substrate, and none of the chiral catalysts is versatile. The search for new chiral catalysts, in particular chiral catalysts with new skeletons, has thus been a perpetual subject in the field of asymmetric catalysis.

Chinese application patent application with a publication number CN108659046A discloses a monophosphine ligand based on a tetramethylspirobiindane skeleton for many chiral catalytic reactions including asymmetric addition, asymmetric hydrogenation, asymmetric coupling and asymmetric allylic alkylation.

Tertiary amine-squaramide bifunctional organocatalysts with multiple hydrogen bond donors have various catalytic uses, and tertiary amine-squaramide bifunctional organocatalysts of different skeletons are reported in the literature and successfully applied to various asymmetric reactions; related literatures include: (a) Applications of Chiral Squaramides: From Asymmetric Organocatalysis to Biologically Active Compounds. Chem. Rec. 2016, 16, 897. (b) Squaramide-Catalyzed Asymmetric Reactions. Chem. Rec. 2017, 17, 994. (c) Recent Advances in Squaramide-Catalyzed Asymmetric Mannich Reactions. Adv. Synth. Catal. 2020, 362, 4487. (d) Squaramide-Based Catalysts in Organic Synthesis (A Review). Russ. J. Gen. Chem. 2022, 92, 287.

However, chiral bifunctional tertiary amine/squaramide catalysts are highly dependent on the reaction substrate when catalyzing the same reaction, see the literature: Organocatalytic Asymmetric Domino Michael/Acyl Transfer Reaction between γ/δ-Hydroxyenones and α-Nitroketones. J. Org. Chem. 2018, 83, 5301; Organocatalytic Enantioselective Cycloaddition of O-Quinone Methides with Oxazolones: Asymmetric Synthesis of Dihydrocoumarins. ChemistrySelect, 2020, 5, 13259. There is therefore a great need to develop more chiral bifunctional tertiary amine/squaramide catalysts with new skeletons.

At the same time, the asymmetrically catalyzed conjugate addition reaction of a 3-trifluoroethylidene indolinone derivative and a pyrazol-3-one is more challenging; the asymmetric addition product, a trifluoromethylated indolin-2-one derivative, has a wide range of applications in medicine, chemical engineering and the like. It is important to provide more choices of efficient chiral catalysts to enable diverse synthesis of chiral trifluoromethylated indolin-2-one derivatives.

### SUMMARY

The present application provides a chiral amine-squaramide compound based on a spirobiindane skeleton that can asymmetrically catalyze the asymmetric conjugate addition reaction of indolinone derivatives and pyrazol-3-ones.

A chiral amine-squaramide compound of formula (I) based on a spirobiindane skeleton:

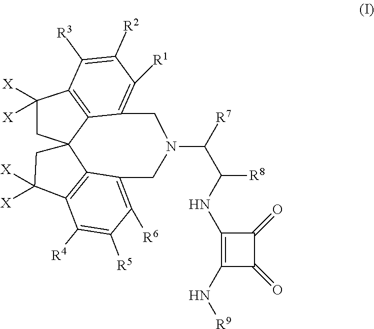

where,

X is H, CH_{3;}

R¹, R⁶, R³, R⁴ are H;

R², R⁵ are H or CH_{3;}

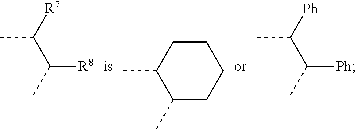

a moiety

R⁹ is phenyl, substituted phenyl, phenylmethylene or phenylethylidene, the substituted phenyl is 1-5 membered replaced with substituents selected from F, CI, Br, I, NO₂, CN, C₁₋₄ alkyl, C₁₋₄ perfluoroalkyl, C₁₋₄ alkoxy, C₁₋₄ perfluoroalkoxy,

an enantiomer thereof, a racemate thereof or a diastereomer thereof.

Further, R⁹ is preferably

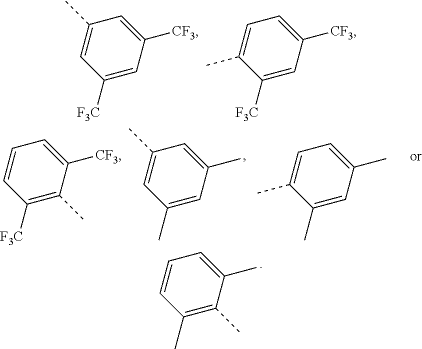

Further, the chiral amine-squaramide compound of formula (I) is:

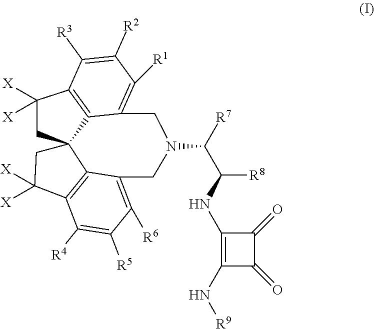

where R¹, R², R³, R⁴, R⁵, R⁶, R⁷, R⁸, R⁹ and X are defined as previously.

Further, the chiral amine-squaramide compound of formula (I) is:

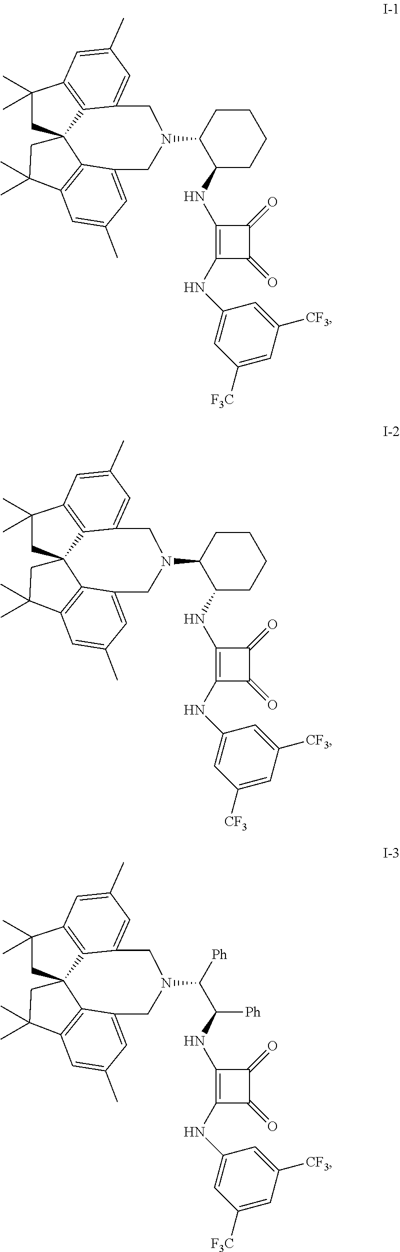

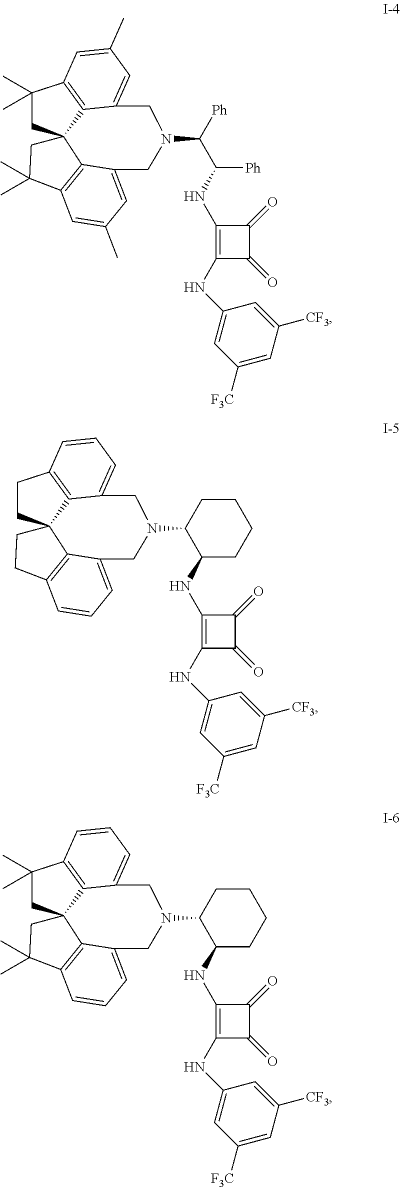

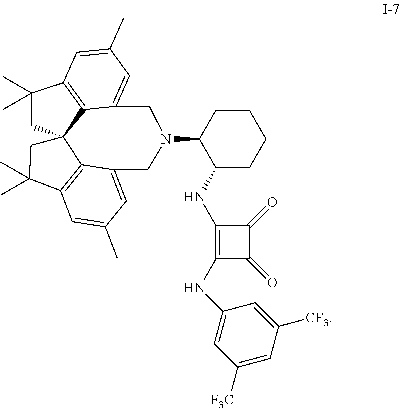

The present application further provides a method for the preparing the chiral amine-squaramide compound of formula (I), including the following step: allowing a compound of formula (II) and a compound of formula (II-e) to have an addition reaction in an organic solvent to obtain a compound of formula (I):

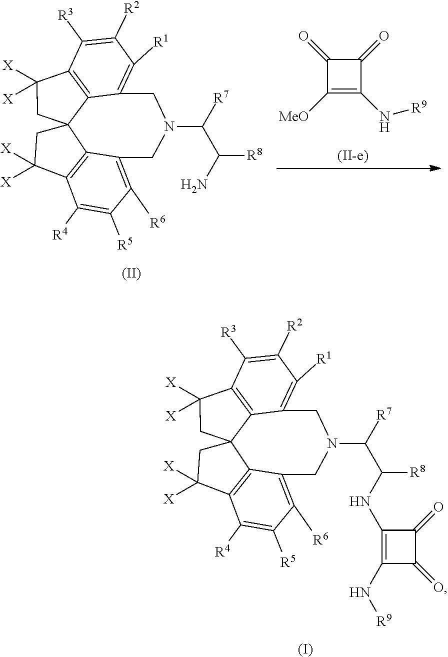

where X, R¹, R², R³, R⁴, R⁵, R⁶, R⁷, R⁸ and R⁹ are as defined previously.

The organic solvent is dichloromethane, chloroform or tetrahydrofuran.

The conditions of the addition reaction are reacting at 0-60° C. with stirring for 1-36 hours.

The chiral amine-squaramide compound based on a spirobiindane skeleton of formula (I) is used in an asymmetrically catalyzed addition reaction.

The asymmetrically catalyzed addition reaction is an asymmetrically catalyzed conjugate addition reaction of a 3-trifluoroethylidene indolinone derivative and a pyrazol-3-one.

It should be understood that the various features of the present application described above and those specifically described in the embodiments below can be combined with each other within the scope of the present application to form new or preferred embodiments. Limited to the space, this will not be repeated here.

The beneficial effects of the present application are mainly embodied in the following aspects:

(1) The chiral amine-squaramide compound based on a spirobiindane skeleton represented by formula (I) of the present application is useful for catalyzing organic reactions with good catalytic activity or enantioselectivity effect, and has economic utility and promising industrial application.

(2) The chiral amine-squaramide compound based on a spirobiindane skeleton represented by formula (I) of the present application is used for asymmetric catalysis of an asymmetric conjugate addition reaction of a 3-trifluoroethylidene indolinone derivative and pyrazol-3-one, and the activity of the catalyst far surpasses the effect of known catalysts, i.e. the asymmetric conjugate addition reaction of the 3-trifluoroethylidene indolinone derivative and pyrazol-3-one can still be asymmetrically catalyzed with 1% of the catalyst, obtaining a high yield and a high enantioselectivity.

(3) The chiral amine-squaramide compound based on a spirobiindane skeleton represented by the formula (I) of the present application uses spirobiindanediol which is inexpensive and readily available as the material, and the synthesis reaction route is simple and easy for large-scale applications.

### DEFINITIONS AND EXPLANATIONS

As used herein, the following terms and phrases, unless otherwise stated, are intended to have the following meanings. A particular term or phrase should not be interpreted as indefinite or unclear without special definition, but rather should be interpreted in its ordinary sense. When a trade name appears herein, it is intended to refer to its corresponding commercial product or its active ingredient. The term “pharmaceutically acceptable” is employed herein with respect to those compounds, materials, compositions, and/or dosage forms. They are, within the scope of sound medical judgment, suitable for use in contact with the tissues of human beings and animals without excessive toxicity, irritation, allergic response, or other problem or complication, commensurate with a reasonable benefit/risk ratio.

The compound of the present application may exist in particular geometric or stereoisomeric forms. The present application contemplates all such compounds, including cis-and trans-isomers, (−)-and (+)-enantiomers, (R)-and (S)-enantiomers, diastereomers, (D)-isomers, (L)-isomers, and the racemic and other mixtures thereof, such as enantiomerically or diastereomerically enriched mixtures, all of which fall within the scope of the present application. Additional asymmetric carbon atoms may be present in substituents such as alkyl groups. All such isomers, as well as mixtures thereof, are included within the scope of the present application. Unless otherwise indicated, the term “cis-trans isomers” or “geometric isomers” result from the inability to rotate freely due to double bonds or single bonds of ring-forming carbon atoms.

Unless otherwise indicated, the term “diastereomer” refers to stereoisomers in which molecules have two or more chiral centers and the relationship between the molecules is non-mirror.

Unless otherwise indicated, the term “(+)” means dextrorotatory, “(−)” means levorotatory, and “(±)” means racemic.

The compound of the present application is named according to the conventional nomenclature in the art or using ChemDraw® software, and the commercially available compounds use supplier catalogs. The relevant sources of raw materials can be commercial reagents or synthesized by reference to the relevant literatures, such as (a)Synthesis and Application of Hexamethyl-1,1′-spirobiindane-Based Phosphine-Oxazoline Ligands in Ni-Catalyzed Asymmetric Arylation of Cyclic Aldimines. J. Org. Chem. 2018, 83, 4034; (b) Synthesis and Application of A New Hexamethyl-1,1′-spirobiindane-based Chiral Bisphosphine (HMSI-PHOS) Ligand in Asymmetric Allylic Alkylation. Org. Biomol. Chem. 2018, 16, 2239; (c)Iron-Catalyzed Enantioselective Si-H Bond Insertions. Org. Lett.2018, 20, 6544; (d) Rhodium-Catalyzed Asymmetric Addition of Organoboronic Acids to Aldimines Using Chiral Spiro Monophosphite-Olefin Ligands: Method Development and Mechanistic Studies. J. Org. Chem. 2018, 83, 11873; (e) Synthesis and Optical Resolution of 3,3,3′, 3′-Tetramethyl-1,1′-spirobiindane-7,7′-diol. Synthesis 2018, 51, 557; (f) Atroposelective Phosphoric Acid Catalyzed Three-Component Cascade Reaction: Highly Enantioselective Synthesis of Axially Chiral N-Arylindoles Angew. Chem. Int. Ed. 2019, 58, 15824; (g)Iron-catalyzed asymmetric intramolecular cyclopropanation reactions using chiral tetramethyl-1,1′-spirobiindane-based bisoxazoline (TMSI-BOX) ligands Org. Biomol. Chem. 2019, 17, 1154; (h)Preparation and application of bisoxazoline ligands with a chiral spirobiindane skeleton for asymmetric cyclopropanation and allylic oxidation Tetrahedron: Asymmetry 17 (2006) 634; (i) Synthesis and application of chiral spiro Cp ligands in rhodium-catalyzed asymmetric oxidative coupling of biaryl compounds with alkenes. J. Am. Chem. Soc. 2016, 138, 5242.

### DESCRIPTION OF EMBODIMENTS

The present application will now be described in detail by way of examples, which are intended to be illustrative of the present application and are not intended to be limiting. The compound of the present application can be prepared by a variety of synthetic methods well known to those skilled in the art, including the specific embodiments set forth below, embodiments formed by combinations thereof with other compound synthesis methods, and equivalents thereof well known to those skilled in the art, and are also commercially available. Preferred embodiments include, but are not limited to, the examples of the present application. It will be apparent to those skilled in the art that various changes and modifications may be made to the specific embodiments of the application without departing from the spirit and scope of the application.

Example 1 Synthesis of (Rₐ,R,R)-II-1.

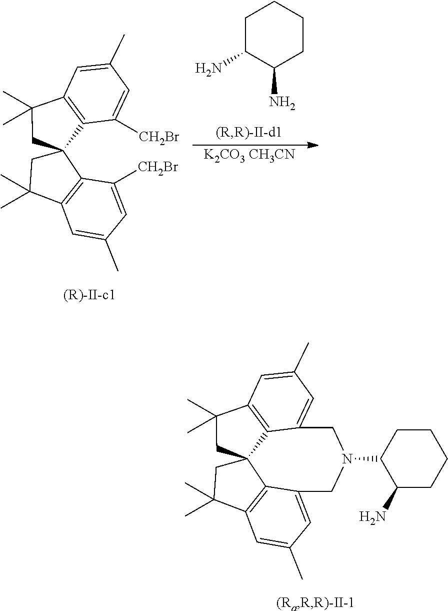

(R)-II-c1 (1.56 g, 3.2 mmol) and (R,R)-II-d1 (12.8 mmol, (R,R)-1,2-diaminocyclohexane) and K₂CO₃ (1.33 g, 9.6 mmol) were dissolved in 70 mL of acetonitrile and heated at reflux under nitrogen for 12 h, then cooled to room temperature and the solvent was removed under reduced pressure. The residue was added to 50 mL of diethyl ether and washed with water. The organic phase was dried and concentrated and subjected to column chromatography (EtOAc/PE=⅙ additional 5% triethylamine was added) to give (Rₐ,R,R)-II-1l (737 mg, with a yield of 52%) as a white solid.

M.p. 84-86° C.; [α]_{D}²⁰=+183.9 (c=1.00, CH₂Cl₂). ¹H NMR (400 MHZ, CDCl₃) δ 6.87 (d, J=8.4 Hz, 4H), 3.85 (d, J=12.9 Hz, 2H), 3.26 (d, J=13.1 Hz, 5H), 2.97-2.85 (m, 1H), 2.49 (t, J=9.8 Hz, 1H), 2.36 (d, J=15.9 Hz, 2H), 2.34 (s, 6H), 2.12 (d, J=9.6 Hz, 1H), 1.90 (d, J=12.5 Hz, 2H), 1.66 (s, 2H), 1.49 (s, 6H), 1.24 (s, 6H), 1.19-1.12 (m, 2H) ppm; HRMS (ESI⁺) calcd for [C₃₁H₄₃N₂]⁺, m/z 443.3421, found 443.3421.

### Example 2 Synthesis of (Rₐ,S,S)-II-2.

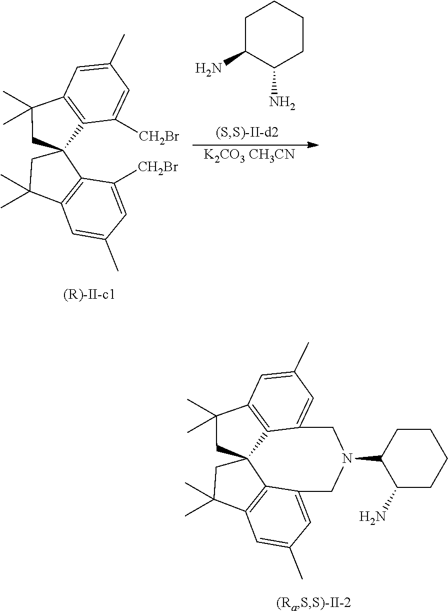

Referring to the procedure of Example 1, (R,R)-II-d1 was replaced with (S,S)-II-d2 (12.8 mmol, (R,R)-1,2-diaminocyclohexane) to give (Rₐ,S,S)-II-2 (790 mg, with a yield of 56%).

M.p. 88-90° C.; [α]_{D}²⁰=+201.2(c=1.00, CH₂Cl₂); HRMS (ESI⁺) calcd for C₃₁H₄₂N₂, m/z 443.3421, found 443.3422.

### Example 3 Synthesis of (Rₐ,R,R)-II-3.

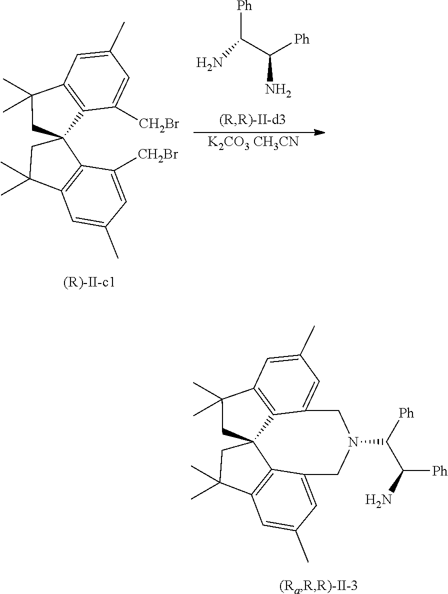

Referring to the procedure of Example 1, (R,R)-II-d1 was replaced with (R,R)-II-d3 (12.8 mmol, (R,R)-1,2-diamino-1, 2-diphenylethane) to give (Rₐ,R,R)-II-3 (886 mg, with a yield of 51%).

M.p. 96-99° C.; [α]_{D}²⁰=+58.2(c=1.00, CH₂Cl₂); HRMS (ESI⁺) calcd for [C₃₉H₄₄N₂+H]⁺, m/z 541.3512, found 541.3573.

### Example 4 Synthesis of (Rₐ,S,S)-II-4.

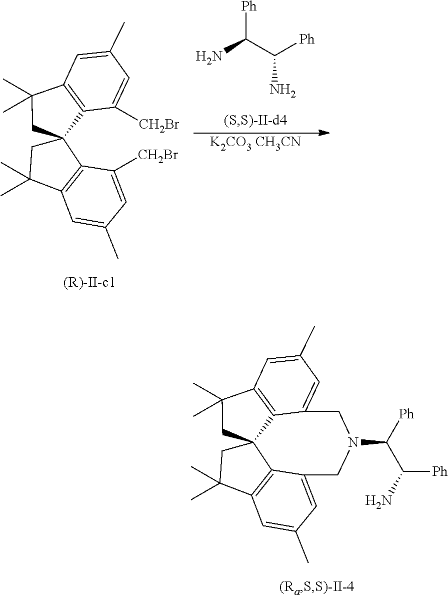

Referring to the procedure of Example 1, (R,R)-II-d1 was replaced with (S,S)-II-d4 (12.8 mmol, (S,S)-1,2-diamino-1,2-diphenylethane) to give (Rₐ,S,S)-II-4 (743 mg, with a yield of 43%).

M.p. 98-102° C.; [α]_{D}²⁰=+17.4 (c=1.00, CH₂Cl₂); HRMS (ESI⁺) calcd for [C₃₉H₄₄N₂+H]⁺, m/z 541.3512, found 541.3577.

### Example 5 Synthesis of (Rₐ,R,R)-I-1.

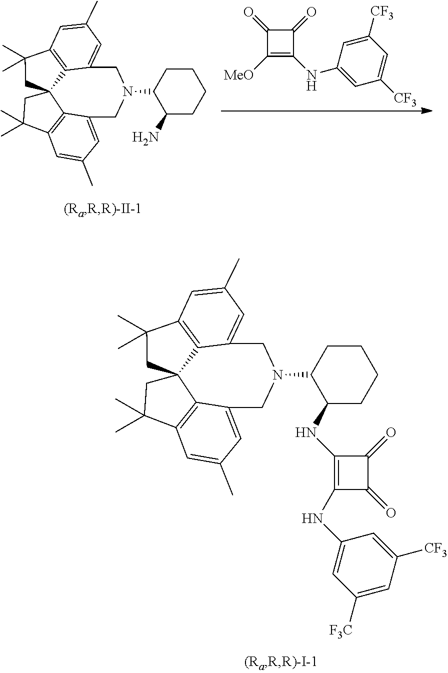

(Rₐ,R,R)-II-1(0.2mmol) was dissolved in 5 mL of dichloromethane and 3-((3, 5-bis (trifluoromethyl) phenyl) amino)-4-methoxycyclobut-3-ene-1, 2-dione (67.8 mg, 0.2 mmol) was added. The mixture was stirred for reaction at room temperature for 48 h, and then filtered and washed with acetonitrile to give the product (Rₐ,R,R)-I-1 (139 mg, with a yield of 93%).

M.p. 234-236° C.; [α]_{D}²⁰=62.5(c=1.00, CH₂Cl₂); ¹H NMR (600 MHz, DMSO-d₆) δ10.09 (s, 1H), 8.02 (s, 2H), 7.67 (s, 1H), 7.63 (s, 1H), 6.81 (d, J=10.6 Hz, 3H), 4.23 (s, 1H), 3.59 (d, J=13.0 Hz, 2H), 3.29 (d, J=13.0 Hz, 2H), 2.72-2.65 (m, 1H), 2.50 (d, J=1.5 Hz, 6H), 2.31-2.23 (m, 2H), 2.19 (s, 4H), 2.03 (d, J=12.8 Hz, 1H), 1.67 (dd, J=36.9, 12.7 Hz, 3H), 1.57 (d, J=7.9 Hz, 1H), 1.40 (s, 6H), 1.34-1.22 (m, 2H), 1.14 (s, 6H) ppm; HRMS (ESI⁺) calcd for [C₄₃H₄₆F₆N₃O₂]⁺, m/z 750.3489, found 750.3489.

### Example 6 Synthesis of (Rₐ,S,S)-I-2.

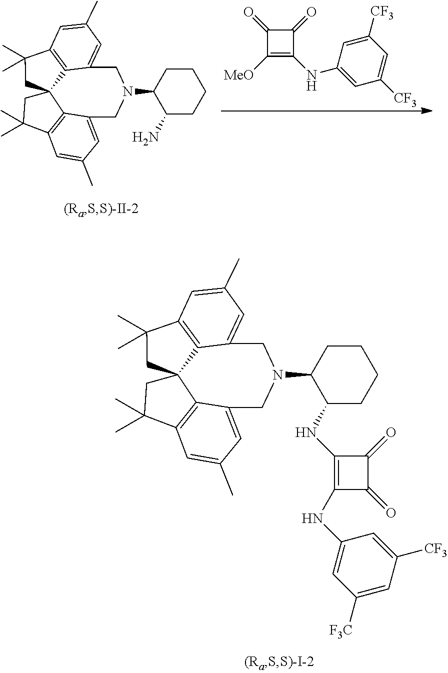

Following the procedure of Example 5, (Rₐ,R,R)-II-1(0.2 mmol) was replaced with (Rₐ,S,S)-II-2 to give (Rₐ,S,S)-I-2 (133 mg, with a yield of 89%).

HRMS (ESI⁺) calcd for [C₄₃H₄₆F₆N₃O₂]⁺, m/z 750.3489, found 750.3487.

### Example 7 Synthesis of (Rₐ,R,R)-I-3.

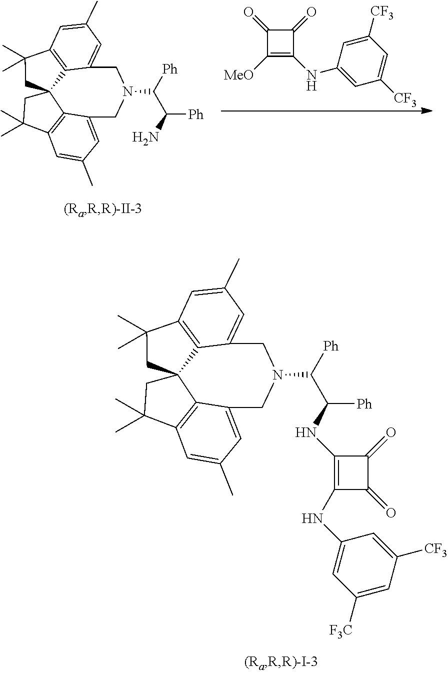

Following the procedure of Example 5, (Rₐ,R,R)-II-1(0.2 mmol) was replaced with (Rₐ, R,R)-II-3 to give (Rₐ, R,R)-I-3 (737 mg, with a yield of 87%).

HRMS (ESI⁺) calcd for [C₅₁H₄₇F₆N₃O₂+H]⁺, m/z 848.3606, found 848.3605.

### Example 8 Synthesis of (Rₐ,S,S)-I-4.

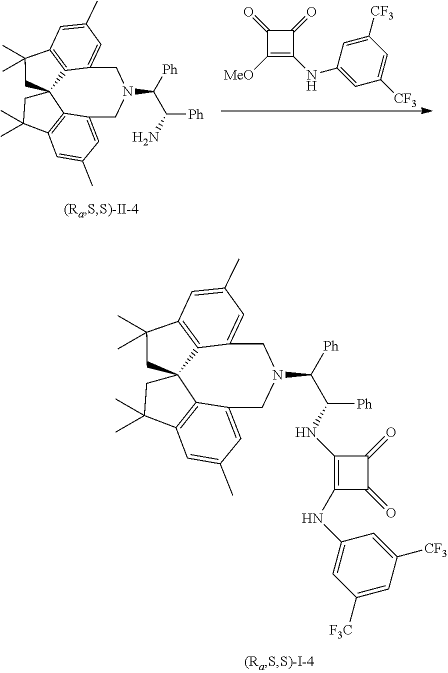

Following the procedure of Example 5, (Rₐ,S,S)-II-1(0.2 mmol) was replaced with (Rₐ,S,S)-II-4 to give (Rₐ,S,S)-I-4 (770 mg, with a yield of 91%).

HRMS (ESI+) calcd for [C51H47F6N302+H] +, m/z 848.3606, found 848.3608.

### Application Example 1

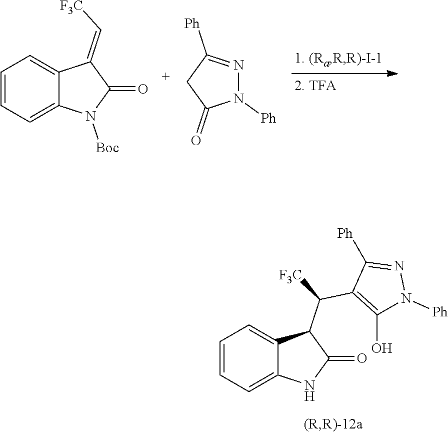

Tert-butyl (E)-2-oxo-3-(2,2,2-trifluoroethylidene) indoline-1-carboxylate (37.6 mg, 0.12 mmol, 1.2 eq) and 2, 5-diphenyl-2, 4-dihydro-3H-pyrazol-3-one (0.1 mmol, 1 eq) were dissolved in 1 mL of 1, 2-dichloroethane, and the organocatalyst (Rₐ,R,R)-I-1 (0.01 mmol, 0.1 eq) was added; then the mixture was stirred and reacted at room temperature for 16 h until the reaction was completed, after which trifluoroacetic acid (1 mmol, 10 eq) was added and reacted for 2 hours, followed by isolation and purification of the resulting asymmetric addition product (R,R)-12a (with a yield of 91%, 95% ee, 94:6 d.r.) by using a preparative chromatography plate (ethyl acetate: n-hexane=1:4).

¹H NMR (600 MHZ, CDCl₃) δ12.20 (s, 1H), 8.49 (s, 1H), 7.92 (d, J=7.9 Hz, 2H), 7.61 (d, J=7.3 Hz, 2H), 7.53 (t, J=7.4 Hz, 2H), 7.50-7.42 (m, 3H), 7.29 (dd, J=14.7, 7.4 Hz, 2H), 7.12 (t, J=7.4 Hz, 1H), 7.08 (d, J=7.2 Hz, 1H), 6.91 (d, J=7.7 Hz, 1H), 4.32 (q, J=9.3 Hz, 1H), 4.05 (s, 1H) ppm.

HPLC conditions: chiral chromatographic column IC-3 (hexane/i-PrOH=95/5, flow rate 1.5 mL/min, λ=210 nm), t_{R}((R,R)-12a)=8.28 min, t_{R}((S,S)-12a)=6.60 min.

### Comparative Example

Referring to the procedure of Application Example 1, (Rₐ,R,R)-I-1 was replaced with the quinine amine-based chiral ammonia-squaramide catalyst (compound 2, configuration (R)) of the literature Mechanochemically Activated Asymmetric Organocatalytic Domino Mannich Reaction-Fluorination. ACS Sustain. Chem. Eng.2020, 8, 14417., to give an asymmetric addition product (S,S)-12a (with a yield of 63%, 88% ee, 97:3 d.r.).

Wherein the structure of compound 2 is as follows:

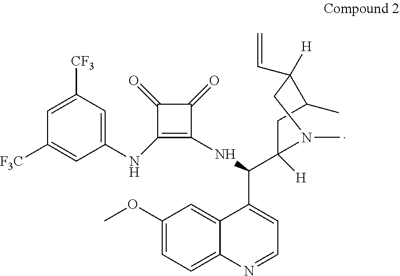 [SMILES: C=CC1CN(C)C([C@H](Nc2c(Nc3cc(C(F)(F)F)cc(C(F)(F)F)c3)c(=O)c2=O)c2ccnc3ccc(OC)cc23)CC1C]

### Application Example 2

Starting materials were charged according to the procedure of Application Example 1, but the amount of the catalyst was reduced, i.e., an organic catalyst (Rₐ,R,R)-I-1 (0.001 mmol, 0.01 eq) was used; then after reaction under stirring at room temperature for 16 h, trifluoroacetic acid (1 mmol, 10eq) was added to react for 2 h, followed by isolation and purification of the resulting asymmetric addition product (R,R)-12a (with a yield of 67%, 88% ee, 96:4 d.r.) by directly using a preparative chromatography plate (ethyl acetate: n-hexane=1:4).

Conclusion: the activity of the catalyst far surpasses the effect of known catalysts, i.e., with 1% of the catalyst, the asymmetric conjugate addition can still be catalyzed, obtaining a high yield and a high enantioselectivity.

### Application Example 3

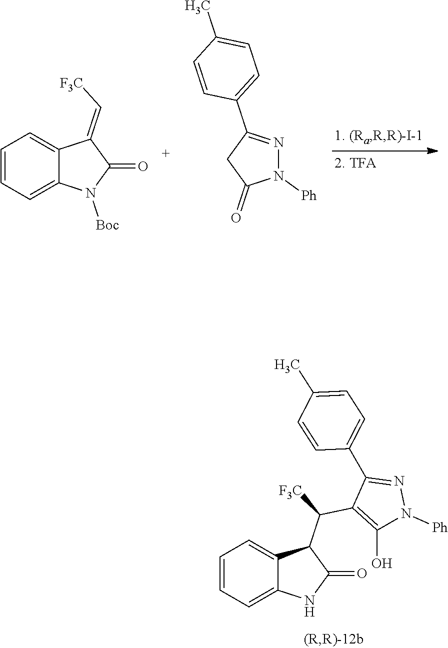 [SMILES: CC(C)(C)OC(=O)N1C(=O)/C(=C/C(F)(F)F)c2ccccc21 ; Cc1ccc(C2=NN(c3ccccc3)C(=O)C2)cc1 ; Cc1ccc(-c2nn(-c3ccccc3)c(O)c2[C@@H]([C@H]2C(=O)Nc3ccccc32)C(F)(F)F)cc1]

Referring to the procedure of Application Example 1, 2,5-diphenyl-2,4-dihydro-3H-pyrazol-3-one was replaced with 2-phenyl-5-(p-tolyl)-2,4-dihydro-3H-pyrazol-3-one to give an asymmetric addition product (R,R)-12b (with a yield of 74%, 93% ee,>99:1 d.r.).

¹H NMR (400 MHz, CDCl₃) δ12.12 (s, 1H), 7.96 (s, 1H), 7.91 (d, J=7.7 Hz, 2H), 7.47 (dd, J=17.6, 8.0 Hz, 4H), 7.32 (dd, J =18.3, 7.7 Hz, 4H), 7.19-7.10 (m, 2H), 6.96 (d, J=7.7 Hz, 1H), 4.33 (q, J=9.7 Hz, 1H), 4.06 (s, 1H), 2.46 (s, 3H) ppm.

HPLC conditions: chiral chromatographic column IC-3 (hexane/i-PrOH= 90/10, flow rate 1 mL/min, λ=254 nm), t_{R} ((R,R)-12b)=7.91 min, t_{R} ((S,S)-12b)=6.56 min.

### Application Example 4

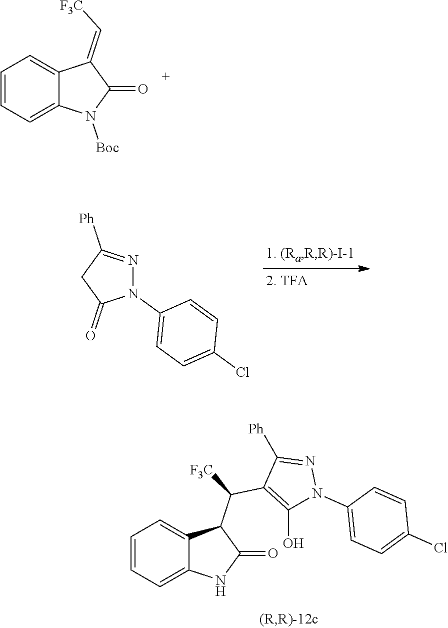 [SMILES: CC(C)(C)OC(=O)N1C(=O)/C(=C/C(F)(F)F)c2ccccc21 ; O=C1CC(c2ccccc2)=NN1c1ccc(Cl)cc1 ; O=C1Nc2ccccc2[C@H]1[C@H](c1c(-c2ccccc2)nn(-c2ccc(Cl)cc2)c1O)C(F)(F)F]

Referring to the procedure of Application Example 1, 2,5-diphenyl-2,4-dihydro-3H-pyrazol-3-one was replaced with 2-(4-chlorophenyl)-5-phenyl-2, 4-dihydro-3H-pyrazol-3-one to give an asymmetric addition product (R,R)-12c (with a yield of 66%, 90% ee, 96:4 d.r.).

1H NMR (400 MHZ, CDCI3) 8 8.34 (s, 1 H), 7.77 (d, J=8.8 Hz, 2H), 7.60-7.52 (m, 5H), 7.45 (d, J=8.8 Hz, 2H), 7.32 (t, J=7.7 Hz, 2H), 7.16 (t, J=7.5 Hz, 1 H), 7.06 (d, J=7.5 Hz, 1 H), 6.97 (d, J=7.8 Hz, 1 H), 4.28 (q, J=8.8 Hz, 1 H), 4.06 (s, 1 H) ppm.

HPLC conditions: chiral chromatographic column IC-3 (hexane/i-PrOH= 95/5, flow rate 1 mL/min, λ=254 nm), t_{R} ((R,R)−12c)=7.09 min, t_{R}((S,S)-12c)=6.02 min.

### Application Example 5

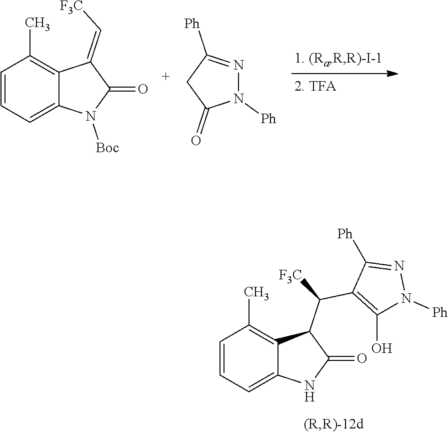

Referring to the procedure of Application Example 1, tert-butyl (E)-2-oxo-3-(2, 2, 2-trifluoroethylidene) indoline-1-carboxylate was replaced with tert-butyl (E)-4-methyl-2-oxo-3-(2,2,2-trifluoroethylidene) indoline-1-carboxylate to give an asymmetric addition product (R,R)-12d (with a yield of 52%,>99% ee, 94:6 d.r.).

¹H NMR (400 MHZ, CDCl₃) δ8.13 (s, 1H), 7.84 (d, J=7.9 Hz, 2H), 7.63-7.44 (m, 7H), 7.34 (t, J=7.4 Hz, 1H), 6.94 (s, 1H), 6.77 (s, 1H), 4.27 (dd, J=18.4, 9.0 Hz, 1H), 4.03 (s, 1H), 2.36 (s, 3H) ppm.

HPLC conditions: chiral chromatographic column IC-3 (hexane/i-PrOH= 90/10, flow rate 1 mL/min, λ=254 nm), t_{R}((R,R)-12d)=4.12 min, t_{R}((S,S)-12d)=6.18 min.

What is claimed is:

1. A chiral amine-squaramide compound of formula (I) based on a spirobiindane skeleton: 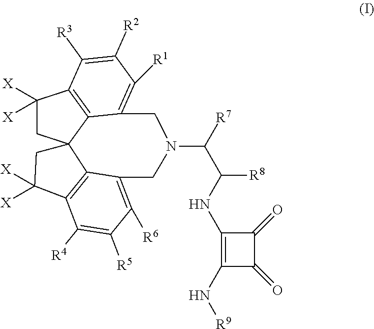
wherein,
X is H, CH_{3;}
R¹, R⁶, R³, R⁴ are H;
R², R⁵ are H or CH₃;
a moiety 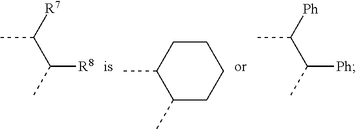
R⁹ phenyl, substituted phenyl, phenylmethylene or phenylethylidene, the substituted phenyl is 1-5 membered replaced with substituents selected from F, CI, Br, I, NO₂, CN, C₁₋₄ alkyl, C₁₋₄ perfluoroalkyl, C₁₋₄ alkoxy, C₁₋₄ perfluoroalkoxy,
an enantiomer thereof, a racemate thereof or a diastereomer thereof.

2. The chiral amine-squaramide compound of formula (I), an enantiomer thereof, a racemate thereof or a diastereomer thereof according to claim 1, wherein the chiral amine-squaramide of formula (I) is:

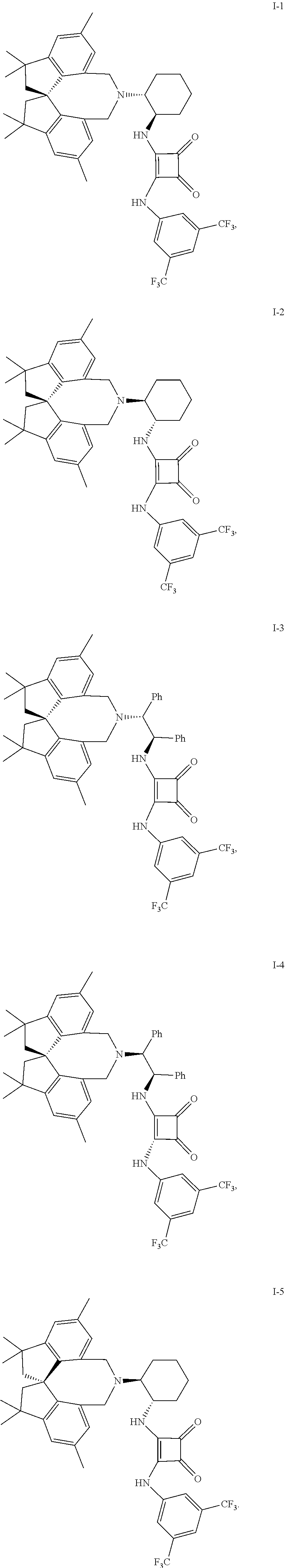

3. A method for preparing the chiral amine-squaramide compound of formula (I) according to claim 1, comprising the following step: allowing a compound of formula (II) and a compound of formula (II-e) to have an addition reaction in an organic solvent to obtain a compound of formula (I): 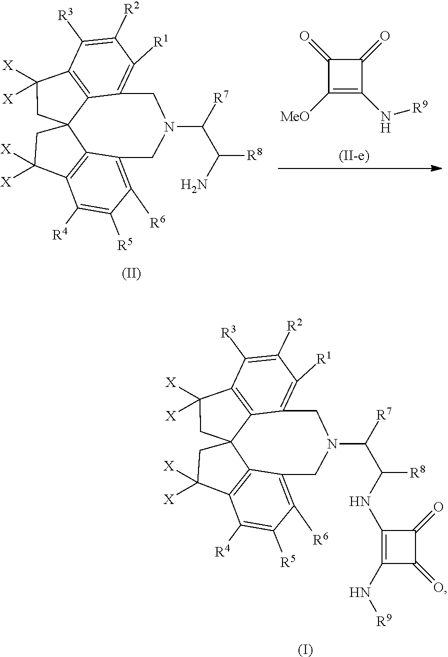
wherein X, R¹, R², R³, R⁴, R⁵, R⁶, R⁷, R⁸ and R⁹ are as defined in claim 1.

4. Use of the chiral amine-squaramide compound of formula (I) based on a spirobiindane skeleton, an enantiomer thereof, a racemate thereof or a diastereomer thereof according to claim 1, wherein the chiral amine-squaramide compound of formula (I) based on a spirobiindane skeleton, an enantiomer thereof, a racemate thereof is used in an asymmetrically catalyzed addition reaction.

5. The use according to claim 4, wherein the asymmetrically catalyzed addition reaction is an asymmetrically catalyzed conjugate addition reaction of a 3-trifluoroethylidene indolinone derivative and a pyrazol-3-one.
